(19) United States
(12) Patent Application Publication
(10) Pub. No.: US 2019/0133532 A1
(43) Pub. Date: May 9, 2019

(54) PRESSURE SENSOR SIGNAL CORRECTION IN A BLOOD PRESSURE MONITORING SYSTEM

(71) Applicant: Edwards Lifesciences Corporation, Irvine, CA (US)

(72) Inventors: Jason A. Wine, Placentia, CA (US); Alexander H. Siemons, Yorba Linda, CA (US); Siddarth Kamath Shevgoor, Laguna Beach, CA (US)

(21) Appl. No.: 16/175,686

(22) Filed: Oct. 30, 2018

Related U.S. Application Data
(60) Provisional application No. 62/582,778, filed on Nov. 7, 2017.

(51) Int. Cl. A61B 5/00; A61B 5/021

(52) U.S. Cl. CPC A61B 5/725; A61B 5/02125; A61B 5/02116

(57) ABSTRACT

Disclosed is a method for filtering a distorted pressure transducer signal from a pressure transducer in a blood pressure monitoring system, comprising: determining one or more pressure transducer signal filtering parameters; receiving a distorted pressure transducer signal from the pressure transducer; filtering the distorted pressure transducer signal to generate a corrected pressure transducer signal based on the one or more pressure transducer signal filtering parameters; and outputting the corrected pressure transducer signal to a patient monitor.

### CROSS-REFERENCE TO RELATED APPLICATIONS

This application claims priority to U.S. Provisional Application No. 62/582,778, filed Nov. 7, 2017, the contents of which are incorporated herein by reference in their entirety.

### BACKGROUND

### Field

The present invention relates to blood pressure monitoring systems and, in particular, to filtering distorted blood pressure signals to remove distortions.

### Relevant Background

A disposable pressure transducer (DPT) system (e.g., a blood pressure monitoring system) may be used to continuously measure a patient's blood pressure. A DPT system may be composed of a patient-tubing connection (typically attached to an arterial line or pulmonary artery catheter “PAC”), flexible tubing, and an integral DPT. The tubing is filled with saline, and is attached to the patient. The DPT is positioned at the same height as the phlebostatic axis of the patient. The patient's blood pressure is measured through the tubing system.

A means to sample blood is often included in conjunction with the DPT system. For example, a Venous Arterial blood Management Protection (VAMP) system, which is composed of a reservoir and a sample site, may be used to allow for the sampling of blood through an access port in the tubing system. The reservoir houses the blood saline mixture (or “clearing volume”), which, when opened, allows blood to be sampled from the integral sample site. After all samples are taken, the clearing volume is infused back into the patient, preventing the loss of blood in a critically ill patient.

Unfortunately, the mechanical elements (e.g., long flexible tubing, reservoirs, sample sites, etc.), which aid in the usability of the blood sampling-blood pressure monitoring system (e.g., a VAMP-DPT system) may fundamentally diminish the accuracy of the pressure monitoring system. As the natural frequency of the system decreases, the ability of the system to faithfully reproduce the frequencies included within the patient blood pressure waveform decreases. This may have a significant effect on the reported blood pressure values.

### SUMMARY

Embodiments of the invention may relate to a method for filtering a distorted pressure transducer signal from a pressure transducer in a blood pressure monitoring system, comprising: determining one or more pressure transducer signal filtering parameters; receiving a distorted pressure transducer signal from the pressure transducer; filtering the distorted pressure transducer signal to generate a corrected pressure transducer signal based on the one or more pressure transducer signal filtering parameters; and outputting the corrected pressure transducer signal to a patient monitor.

### BRIEF DESCRIPTION OF THE DRAWINGS

FIG. 1 is a diagram illustrating an example known blood sampling system in the environment of a typical set up in a hospital room and connected to a patient.

FIG. 2 is a block diagram illustrating various example blood pressure waveforms in a known blood pressure monitoring system.

FIG. 3A is a block diagram illustrating an example blood pressure monitoring system diagram according to one embodiment of the invention.

FIG. 3B is a block diagram illustrating various example blood pressure waveforms in a blood pressure monitoring system according to one embodiment of the invention.

FIG. 4 is a block diagram illustrating an example system in which embodiments of the invention may be practiced.

FIG. 5 is a block diagram illustrating an example microprocessor-based filter unit.

FIG. 6 is a flowchart illustrating an example method for filtering a distorted pressure signal.

FIG. 7 is a diagram illustrating an example pressure response curve generated in response to an impulse.

FIG. 8 is a flowchart illustrating an example method for determining one or more pressure transducer signal filtering parameters in situ.

FIG. 9 is a block diagram illustrating an example system incorporating a passive hardware filter, according to one embodiment of the invention.

### DETAILED DESCRIPTION

As has been described, mechanical elements (e.g., long flexible tubing, reservoirs, sample sites, etc.), which aid in the usability of a blood sampling-blood pressure monitoring system (e.g., a VAMP-DPT system) may fundamentally diminish the accuracy of the pressure monitoring system. As the natural frequency of the system decreases, the ability of the system to faithfully reproduce the frequencies included within the patient blood pressure waveform decreases. This may have a significant effect on the reported blood pressure values. Further, the way in which a kit adds distortion may differ significantly from kit to kit. However, for a given configuration, the distortion profile may be repeatable. By using a hardware or software filter, the pressure signal according to the known distortion profile may be corrected for a specific kit, as will be described.

Removing the distortion caused by the mechanical elements in the system allows for improved accuracy and can dramatically increase the usability of certain system kits. For example, kits could be made longer and more flexible; and reservoirs and sample sites could be made softer.

Embodiments of the invention may be directed to a method, apparatus, and system for filtering a pressure transducer signal to remove distortions in the signal. The filtering may be performed by a microprocessor-based filter unit or by a passive hardware filter (e.g., a printed circuit board “PCB” passive hardware filter). In cases where a microprocessor-based filter unit is used, the filtering may be performed based on one or more filtering parameters relating to at least the natural frequency of the system. The filtering parameters for a kit may be determined a priori (e.g., measured at a factory) and entered into the microprocessor-based filter unit on-site (e.g., in a hospital) through one of various means. In a different embodiment, the filtering parameters for a kit may be measured in situ (e.g., in a hospital). These techniques will be further described in detail hereafter.

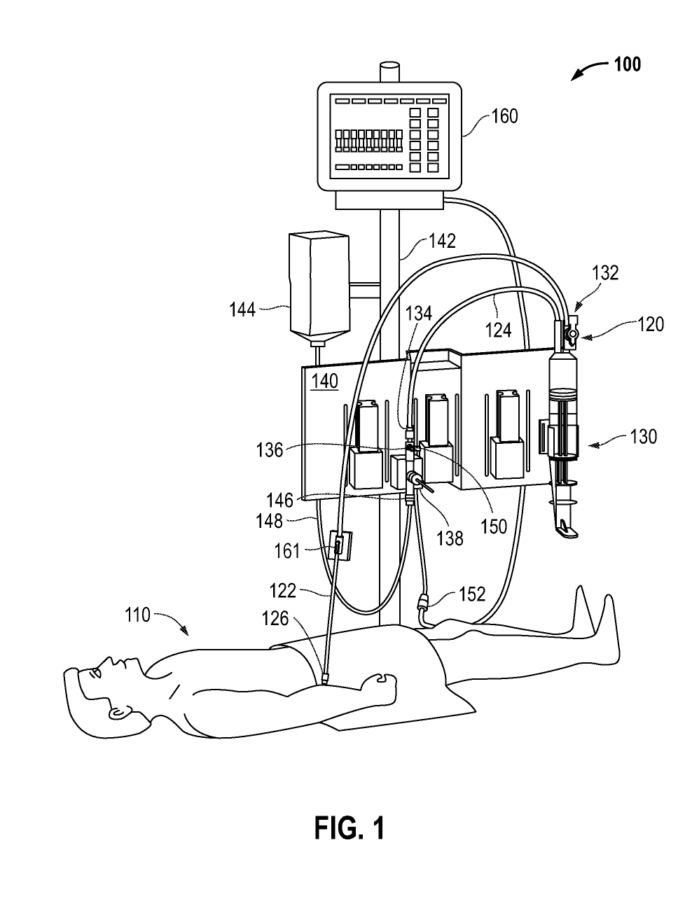

FIG. 1 illustrates an example of a known blood sampling system 120 in an example blood sampling-blood pressure monitoring system 100 as may be set up in a hospital room and connected to a patient 110. The blood sampling system 120 comprises a conduit line having a distal segment 122 toward the patient 110 and a proximal segment 124. The conduit line is primarily medical grade pressure tubing. The distal segment 122 may terminate in a male luer connector 126 for attaching to a female luer connector (not shown) of an injection site, or other conduit leading to the patient 110. A reservoir 130 connects to the conduit line via a three-port control valve 132 interposed between the distal segment 122 and proximal segment 124. The control valve 132 externally resembles a stopcock and controls fluid flow between the conduit line and the reservoir 130.

The proximal segment 124 extends from the control valve 132 and terminates in a female luer connector 134 attached to a stopcock 136 of a pressure transducer 138. The reservoir 130 and pressure transducer 138 removably mount to a bracket 140 which, in turn, may be secured to a conventional pole support 142 with the reservoir 130 in a vertical orientation.

As mentioned above, the blood sampling system 120 forms a portion of the blood sampling-blood pressure monitoring system 100 and the pressure monitoring system portion includes fluid pressure transducer 138. For example, the fluid pressure transducer 138 may be a DPT 138. However, it should be appreciated that any type of pressure transducer may be utilized.

A supply of flush solution 144 connects to a flush port 146 of the transducer 138 via tubing 148. Typically, the flush solution 144 comprises a bag of physiological fluid such as saline surrounded by a pressurized sleeve that squeezes the fluid and forces it through the tubing 148. In addition, an infusion fluid supply (not shown) may be provided in communication with an infusion port 150 of the stopcock 136. The pressure transducer 138 is thus placed in fluid communication with the arterial or venous system of the patient 110 through the conduit line and includes a cable and plug 152 to connect to a suitable display monitor (e.g., patient monitor 160), as will be described in more detail hereafter. Although the pressure transducer 138 is shown positioned in the proximal segment 124, it could also be located in the distal segment 122.

The sampling system 120 may further include a fluid sampling site 161 that desirably defines a Z-shaped flow passage adjacent a pre-slit septum. With this configuration, a minimal amount of flush volume is needed to clear the line after sampling. The septum preferably comprises an elastomeric disc which accepts a blunt cannula and reseals after each sample is drawn, reducing the potential for contamination and eliminating the danger of needle sticks.

Therefore, an example blood sampling-blood pressure monitoring system 100 includes a blood sampling portion 120 in conjunction with a blood pressure monitoring system primarily including pressure transducer 138 (e.g., a DPT) coupled through a cable to patient monitor 160, as will be described in more detail hereafter. However, it should be appreciated that this is just an example, and that any suitable blood sampling-blood pressure monitoring system with a suitable pressure transducer, may be utilized with embodiments of the invention to be hereafter described.

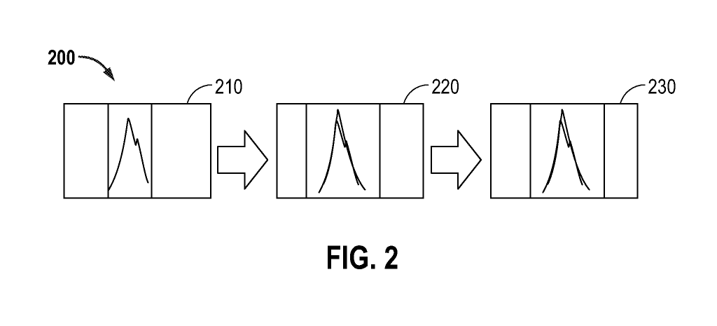

Turning to particular aspects of the invention, with reference to FIG. 2, a block diagram 200 illustrating various example blood pressure waveforms derived from a blood pressure monitoring system is shown. Block 210 comprises the blood pressure waveform at the catheter site, which represents the waveform in its undistorted state. Block 220 comprises the pressure waveform detectable at the pressure transducer. The waveform in block 220 comprises distortions superposed on the true blood pressure waveform, where the distortion are caused by the mechanical elements in the kit (e.g., a kit may include components of the blood sampling-blood pressure monitoring system—tubes, reservoirs, sample sites, etc.). Block 230 comprises the pressure waveform passed onto the patient monitor from the pressure transducer (e.g., as an electronic signal). As can be seen, the waveform in block 230 contains the same distortions as the waveform in block 220. As explained above, removal of the distortions from the waveform in block 230 would allow improved blood pressure measurement accuracy and dramatically increase the usability of certain system kits.

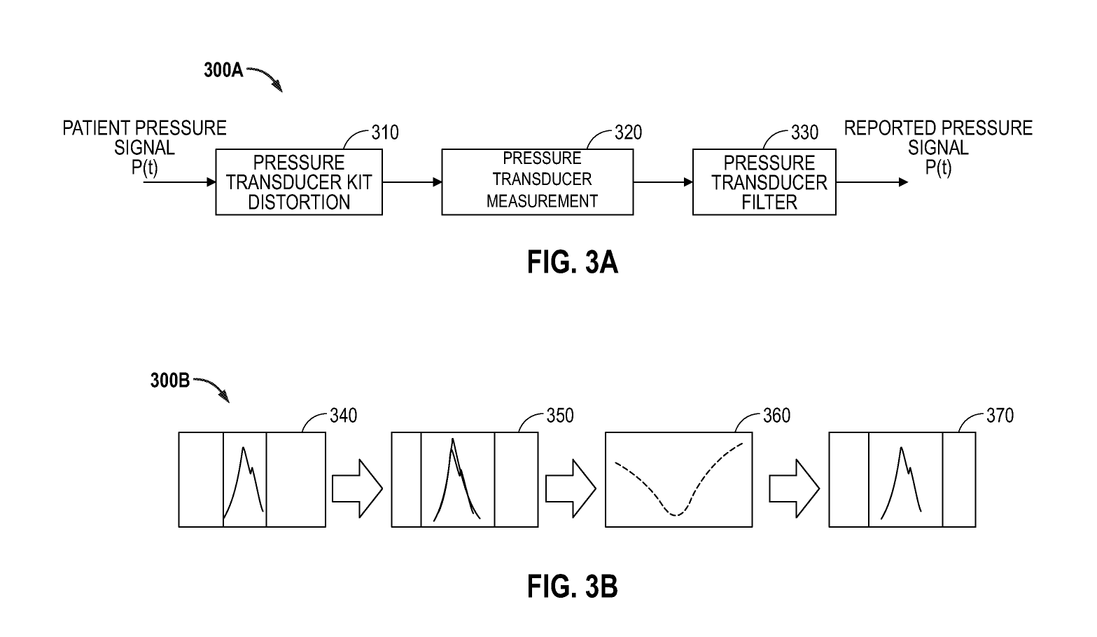

Referring to FIGS. 3A and 3B, FIG. 3A shows a block diagram 300A illustrating an example blood pressure monitoring system diagram according to one embodiment of the invention. FIG. 3B shows a block diagram 300B illustrating various example blood pressure waveforms in a blood pressure monitoring system according to one embodiment of the invention. The patient blood pressure signal (P(t)) in FIG. 3A corresponds to the waveform shown in block 340. A distortion 310 caused by mechanical elements in the kit is superposed onto the patient pressure signal. As a result, the pressure transducer measurement 320 corresponds to a distorted signal. The corresponding distorted waveform is shown in block 350 (e.g., the one different than the one from block 340). The filter unit 330 (e.g., pressure transducer filter) filters the distorted signal in order to remove the distortion. A transfer function representative of the filtering performed by the filter unit 330 is shown in block 360. The reported pressure signal in FIG. 3A, which is the result of the filtering performed by the filter unit 330, should be free from distortions. Ideally, the reported pressure signal should match the patient blood pressure signal. The waveform of the reported pressure signal is shown in block 370, which ideally should match the waveform shown in block 340.

In one embodiment, the filtering of the distorted pressure transducer output signal may be performed by a microprocessor-based filter unit. In different embodiments, the microprocessor-based filter unit may take any of various possible forms. For example, the microprocessor-based filter unit may be in the form of a box or a dongle that is situated between the pressure transducer connector and the patient monitor. The microprocessor-based filter box or dongle may obtain power from either the patient monitor or a separate power outlet. In one embodiment, the microprocessor-based filter may be integrated into the patient monitor. In an additional embodiment, the microprocessor-based filter may be integrated into the cable between the pressure transducer connector and the patient monitor, and obtain power from the patient monitor. It should be appreciated that the above is a non-exhaustive list, and the form of the microprocessor-based filter unit does not limit the invention.

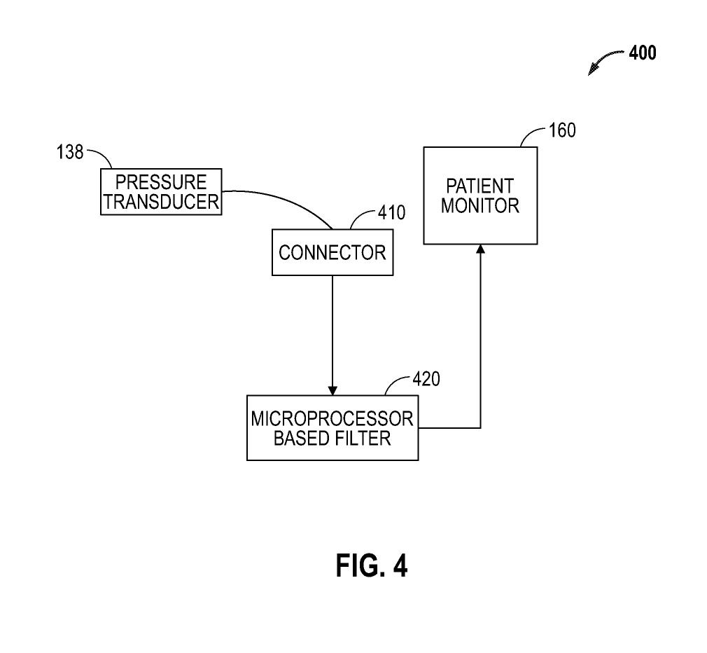

Referring to FIG. 4, a block diagram illustrating an example system 400 in which embodiments of the invention may be practiced is shown. The microprocessor-based filter unit 420 is connected to the pressure transducer (e.g., DPT) 138 by a pressure transducer connector 410 (e.g., plug 152 of FIG. 1) and to the patient monitor 160. For example, the microprocessor-based filter unit 420 may be connected to the DPT 138 and the patient monitor 160 via cables. However, in some embodiments, wireless connections may be utilized. In particular, the outputted pressure signal from the pressure transducer 138 may be transmitted through the connector 410 to the microprocessor-based filter unit 420 where it is filtered to remove distortions. The filtered pressure signal may then be outputted from the microprocessor-based filter unit 420 to the patient monitor 160. The microprocessor-based filter unit 420 may be in the form of a box or a dongle that is situated between the pressure transducer connector 410 and the patient monitor 160. As previously described, the microprocessor-based filter 420 may be a separate hardware/software box, dongle, may be integrated with the patient monitor 160, may be integrated with the cable, etc. It should be appreciated that the microprocessor-based filter 420 may be any sort of computing device to accomplish the described functions and may be in any suitable configuration, the previously described implementations being only examples.

In one embodiment, filtering the pressure signal from the pressure transducer 138 may comprise: assigning parameters to characterize the frequency response of the system (e.g., filter equation); performing a Fast Fourier Transform (FFT) on the pressure signal; dividing all frequencies by the filter equation; and performing an Inverse FFT.

The microprocessor-based filter unit 420 may filter the distorted pressure signal based on one or more filtering parameters. There may exist a large number of unique model numbers for system kits. Different kits vary in their natural frequency and other mechanical characteristics and require different filtering parameters for correctly removing the distortions. Accordingly, the microprocessor-based filter unit 420 may be adaptable and take as inputs the correct filtering parameters that correspond to the particular kit to which it is connected. As has been described, a kit may include various differing components of a blood sampling-blood pressure monitoring system—tubes, reservoirs, sample sites, etc.

In different embodiments, the filtering parameters can be inputted into the microprocessor-based filter unit 420 in various ways. In some embodiments, the filtering parameters may be determined a priori (e.g., measured in a factory) and supplied with the kit (e.g., printed, encoded in a barcode, recorded in a radio-frequency identification “RFID” tag, recorded in a memory module, etc.). Therefore, for example, in the hospital, the filtering parameters can be inputted into the microprocessor-based filter unit 420 manually, with a scan of a barcode, with a read of an RFID tag, or with a read from a memory module. In one embodiment, a memory module recording filtering parameters may be connected to or integrated into the pressure transducer connector 410. It should be appreciated that the way the filtering parameters are inputted into the microprocessor-based filter unit 420 may be done in a variety of different ways, and this list is not all inclusive.

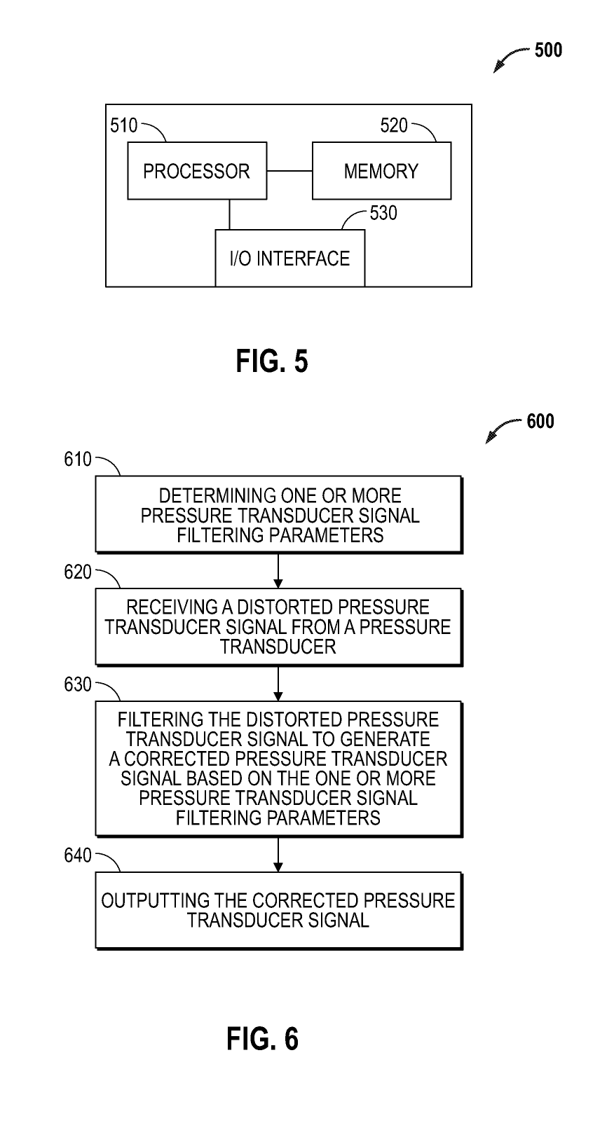

Referring to FIG. 5, a block diagram illustrating an example microprocessor-based filter unit 500 is shown. The microprocessor-based filter unit 500 comprises a processor 510, a memory 520, and an input/output (I/O) interface 530. The memory 520 may store code which, when executed by the processor 510, causes the processor 510 to perform a filtering operation to remove distortions from a pressure signal. The memory 520 may further store filtering parameters that may be used for the filtering operation. The I/O interface 530 may be adapted for receiving distorted a pressure signal from a pressure transducer and for transmitting a filtered pressure signal to a patient monitor. Further, the I/O interface 530 may be implemented with one or more of a manual input device, a barcode scanner, an RFID reader, or a data bus, etc., for receiving filtering parameters. It should be appreciated that interfaces and communications may include wired and wireless components.

Referring to FIG. 6, a flowchart illustrating an example method 600 for filtering a distorted pressure signal is shown. At block 610, one or more pressure transducer signal filtering parameters may be determined. At block 620, a distorted pressure transducer signal may be received from a pressure transducer. At block 630, the distorted pressure transducer signal may be filtered to generate a corrected pressure transducer signal based on the one or more pressure transducer signal filtering parameters. At block 640, the corrected pressure transducer signal may be outputted to a patient monitor.

In another embodiment, the filtering parameters can be measured in situ once the blood sampling-blood pressure monitoring system has been set up (e.g., in a hospital). The DPT may include a flush device that also can be used for sending transient pressure waves through the line. A Snap-Tab device of the DPT may be a rubber tab which when pulled and then released sends a square wave through the pressure column to measure the inherent frequency response of the entire system, which includes the tubing and other mechanical elements. In another embodiment, an automated actuator that is capable of creating an impulse into the system may be implemented. The filtering parameters may be derived based on the inherent frequency response of the system.

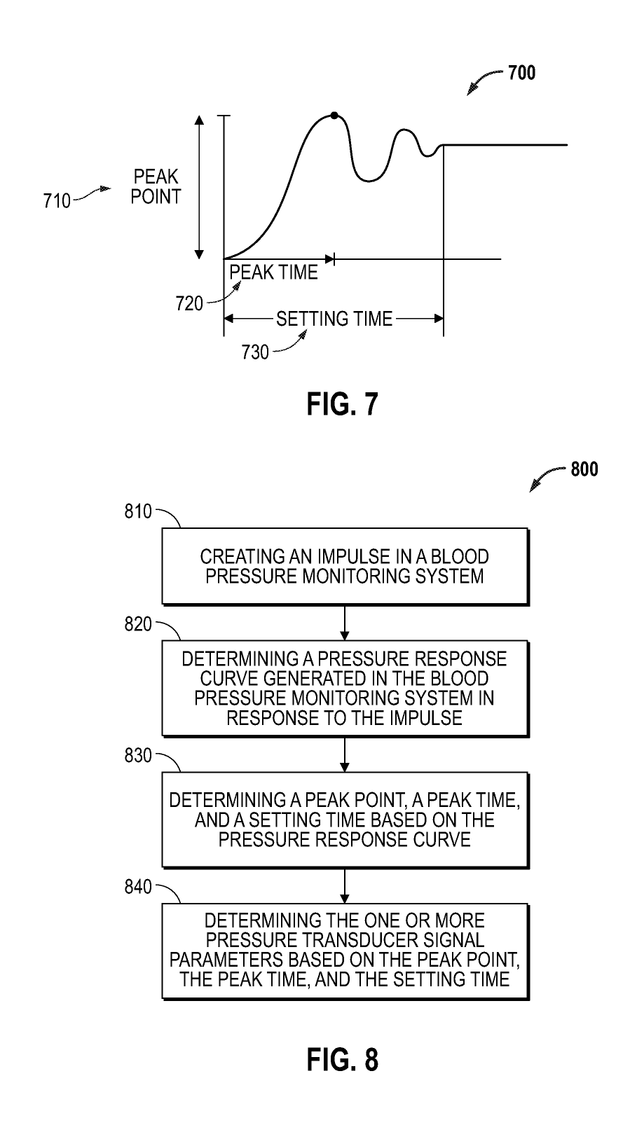

Referring to FIG. 7, a diagram illustrating an example pressure response curve 700 generated in response to an impulse is shown. The Peak Point 710 (PP) is the highest pressure reached in the system. The Peak Time 720 (PT) is time it takes for the pressure in the system to reach the Peak Point 710 after the introduction of the impulse. The Setting Time 730 (Tₛ) is the time it take for the amplitude of the pressure oscillation in the system to be attenuated to a level below a predetermined threshold level. Accordingly, damping and natural frequency ωₙ can be solved for based on the following three formulas:

$$\begin{matrix}PP = 1 + {e}^{- ζπ \sqrt{1 - {ζ}^{2}}} & ( 1 ) \\ PT = \frac{π}{{ω}_{n} \sqrt{1 - {ζ}^{2}}} & ( 2 ) \\ {T}_{s} = \frac{4}{{ζω}_{n}} & ( 3 )\end{matrix}$$

It should be appreciated that damping and natural frequency ωₙ, once known, can be used as filtering parameters in the microprocessor-based filter unit.

Referring to FIG. 8, a flowchart illustrating an example method 800 for determining one or more pressure transducer signal filtering parameters in situ is shown. At block 810, an impulse in a blood pressure monitoring system may be created. At block 820, a pressure response curve generated in the blood pressure monitoring system in response to the impulse may be determined. At block 830, a peak point, a peak time, and a setting time may be determined based on the pressure response curve. At block 840, the one or more pressure transducer signal filtering parameters may be determined based on the peak point, the peak time, and the setting time.

In an additional embodiment, instead of a microprocessor-based filter unit, a passive hardware filter (e.g., a PCB passive hardware filter) may be used to filter the distorted pressure signal outputted by the pressure transducer to remove the distortions. The hardware filter may be tuned to a particular kit. The same passive hardware filter can be used with kits that are similar. In one embodiment, the passive hardware filter may be a passive differentiator that reduces the amplitude response for a given frequency.

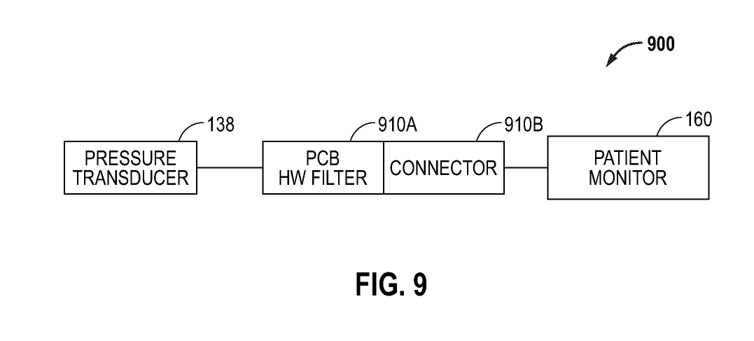

Referring to FIG. 9, a block diagram illustrating an example system 900 incorporating a passive hardware filter, according to one embodiment of the invention, is shown. The passive hardware filter 910A (e.g., a PCB passive hardware filter) may be connected to or incorporated into the pressure transducer connector 910B. Of course, the passive hardware filter 910A can be connected into the system 900 in other locations between the pressure transducer 138 and the patient monitor 160 without deviating from the invention. Therefore, the distorted pressure signal outputted from the pressure transducer 138 is filtered by the passive hardware filter 910A to remove distortions before being fed into the patient monitor 160.

Therefore, embodiments of the invention are related to a method, apparatus, and system for removing distortions in the blood pressure signal outputted by a pressure transducer. The distortions may be caused by mechanical elements in the blood sampling-blood pressure monitoring system. In particular, as has been described, a kit may include components of the blood sampling-blood pressure monitoring system—tubes, reservoirs, sample site, etc. The ability to remove the distortions allows for improved accuracy and dramatically increases the usability of certain system kits. For example, kits could be longer and more flexible; reservoirs and sample sites could be made softer, etc.

The various illustrative logical blocks, processors, modules, and circuitry described in connection with the embodiments disclosed herein may be implemented or performed with a general purpose processor, a specialized processor, circuitry, a microcontroller, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor may be a microprocessor or any conventional processor, controller, microcontroller, circuitry, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module/firmware executed by a processor, or any combination thereof. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor.

Further, embodiments are described in terms of sequences of actions to be performed by, for example, elements of a computing device. It will be recognized that various actions described herein can be performed by specific circuits (e.g., application specific integrated circuits), by program instructions being executed by one or more processors, or by a combination of both. Additionally, these sequences of actions described herein can be considered to be embodied entirely within any form of computer readable storage medium (e.g., non-transitory) having stored therein a corresponding set of computer instructions that upon execution would cause an associated processor to perform the functionality described herein. Thus, the various aspects of the disclosure may be embodied in a number of different forms, all of which have been contemplated to be within the scope of the claimed subject matter.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A method for filtering a distorted pressure transducer signal from a pressure transducer in a blood pressure monitoring system, comprising:
determining one or more pressure transducer signal filtering parameters;
receiving a distorted pressure transducer signal from the pressure transducer;
filtering the distorted pressure transducer signal to generate a corrected pressure transducer signal based on the one or more pressure transducer signal filtering parameters; and
outputting the corrected pressure transducer signal to a patient monitor.

2. The method of claim 1, wherein the one or more pressure transducer signal filtering parameters are determined in situ, and determining the one or more pressure transducer signal filtering parameters comprises:
creating an impulse in the blood pressure monitoring system;
determining a pressure response curve generated in the blood pressure monitoring system in response to the impulse;
determining a peak point, a peak time, and a setting time based on the frequency response curve; and
determining the one or more pressure transducer signal filtering parameters based on the peak point, the peak time, and the setting time.

3. The method of claim 2, wherein the one or more pressure transducer signal filtering parameters comprise a damping value and a natural frequency value.

4. The method of claim 1, wherein determining the one or more pressure transducer signal filtering parameters comprises at least one of: receiving a manual input, scanning a barcode, reading a radio-frequency identification (RFID) tag, or reading a memory module.

5. The method of claim 1, wherein the distorted pressure transducer signal is filtered at a microprocessor-based filter unit.

6. The method of claim 5, wherein the microprocessor-based filter unit obtains power from the patient monitor.

7. The method of claim 5, wherein the microprocessor-based filter unit is integrated into a cable that connects the pressure transducer to the patient monitor.

8. A filtering device to filter a distorted pressure transducer signal from a pressure transducer in a blood pressure monitoring system, the filtering device comprising:
an interface; and
a processor configured to:
determine one or more pressure transducer signal filtering parameters;
receive a distorted pressure transducer signal from the pressure transducer through the interface;
filter the distorted pressure transducer signal to generate a corrected pressure transducer signal based on the one or more pressure transducer signal filtering parameters; and
output, through the interface, the corrected pressure transducer signal to a patient monitor.

9. The filtering device of claim 8, wherein the one or more pressure transducer signal filtering parameters are determined in situ, and the processor determines the one or more pressure transducer signal filtering parameters by:
creating an impulse in the blood pressure monitoring system;
determining a pressure response curve generated in the blood pressure monitoring system in response to the impulse;
determining a peak point, a peak time, and a setting time based on the frequency response curve; and
determining the one or more pressure transducer signal filtering parameters based on the peak point, the peak time, and the setting time.

10. The filtering device of claim 9, wherein the one or more pressure transducer signal filtering parameters comprise a damping value and a natural frequency value.

11. The filtering device of claim 8, wherein determining the one or more pressure transducer signal filtering parameters comprises at least one of the processor: receiving a manual input, receiving a scanned barcode, receiving a radio-frequency identification (RFID) tag, or reading a memory module.

12. The filtering device of claim 8, wherein the distorted pressure transducer signal is filtered by the processor at a microprocessor-based filter unit.

13. The filtering device of claim 12, wherein the microprocessor-based filter unit obtains power from the patient monitor.

14. The filtering device of claim 12, wherein the microprocessor-based filter unit is integrated into a cable that connects the pressure transducer to the patient monitor.

15. A non-transitory computer-readable medium comprising code which, when executed by a processor, causes the processor to perform a method for filtering a distorted pressure transducer signal from a pressure transducer in a blood pressure monitoring system, the method comprising:
determining one or more pressure transducer signal filtering parameters;
receiving a distorted pressure transducer signal from the pressure transducer;
filtering the distorted pressure transducer signal to generate a corrected pressure transducer signal based on the one or more pressure transducer signal filtering parameters; and
outputting the corrected pressure transducer signal to a patient monitor.

16. The non-transitory computer-readable medium of claim 15, wherein the one or more pressure transducer signal filtering parameters are determined in situ, and determining the one or more pressure transducer signal filtering parameters comprises:
creating an impulse in the blood pressure monitoring system;
determining a pressure response curve generated in the blood pressure monitoring system in response to the impulse;
determining a peak point, a peak time, and a setting time based on the frequency response curve; and
determining the one or more pressure transducer signal filtering parameters based on the peak point, the peak time, and the setting time.

17. The non-transitory computer-readable medium of claim 16, wherein the one or more pressure transducer signal filtering parameters comprise a damping value and a natural frequency value.

18. The non-transitory computer-readable medium of claim 15, wherein determining the one or more pressure transducer signal filtering parameters comprises at least one of:
receiving a manual input, scanning a barcode, reading a radio-frequency identification (RFID) tag, or reading a memory module.

19. The non-transitory computer-readable medium of claim 15, wherein the distorted pressure transducer signal is filtered at a microprocessor-based filter unit.

20. The non-transitory computer-readable medium of claim 19, wherein the microprocessor-based filter unit obtains power from the patient monitor.

21. The non-transitory computer-readable medium of claim 19, wherein the microprocessor-based filter unit is integrated into a cable that connects the pressure transducer to the patient monitor.
